(19) United States
(12) Patent Application Publication
(10) Pub. No.: US 2012/0149100 A1
(43) Pub. Date: Jun. 14, 2012

(54) CARDIOMYOCYTE CELL POPULATIONS

(75) Inventors: GORDON KELLER, (US); Chang-Yi Chen, New York, NY (US)

(73) Assignee: Mount Sinai School of Medicine, New York, NY (US)

(21) Appl. No.: 13/398,428

(22) Filed: Feb. 16, 2012

Related U.S. Application Data
(63) Continuation of application No. 11/993,688, filed on Jun. 26, 2009, now abandoned.
(60) Provisional application No. 60/693,537, filed on Jun. 23, 2005.

(51) Int. Cl. C12N 5/071

(52) U.S. Cl. 435/325

(57) ABSTRACT

The present invention provides methods for inducing the differentiation of cardiac progenitor cells and cell populations produced by the methods of the invention. The invention further provides a method of screening for agents that affect cardiomyocytes, and a method of cardiomyocyte replacement therapy.

### CROSS REFERENCE TO RELATED APPLICATION

This application claims priority of U.S. application Ser. No. 60/693,537 filed Jun. 23, 2005, the disclosure of which is incorporated herein by reference.

### STATEMENT REGARDING FEDERALLY FUNDED RESEARCH

This invention was made with government support under Grant No. R01 HL071800 awarded by the National Institutes of Health. The United States government may have certain rights in this invention.

### BACKGROUND OF THE INVENTION

During embryonic development, the tissues of the body are formed from three major cell populations: ectoderm, mesoderm and definitive endoderm. These cell populations, also known as primary germ cell layers, are formed through a process known as gastrulation. Following gastrulation, each primary germ cell layer generates a specific set of cell populations and tissues. Mesoderm gives rise to blood cells, endothelial cells, cardiac and skeletal muscle, and adipocytes. Definitive endoderm generates liver, pancreas and lung. Ectoderm gives rise to the nervous system, skin and adrenal tissues.

The process of tissue development from these germ cell layers involves multiple differentiation steps, reflecting complex molecular changes. With respect to mesoderm and its derivatives, three distinct stages have been defined. The first is the induction of mesoderm from cells within a structure known as the epiblast. The newly formed mesoderm, also known as nascent mesoderm, migrates to different positions that will be sites of future tissue development in the early embryo. This process, known as patterning, entails some molecular changes that are likely reflective of the initial stages of differentiation towards specific tissues. The final stage, known as specification, involves the generation of distinct tissues from the patterned mesodermal subpopulations. Recent studies have provided evidence which suggests that mesoderm is induced in successive waves which represent subpopulations with distinct developmental potential. The mesoderm that is formed first migrates to the extraembryonic region and gives rise to hematopoietic and endothelial cells, whereas the next population migrates anteriorly in the developing embryo and contributes to the heart and cranial mesenchyme. These lineage relationships were defined initially through histological analysis and have been largely confirmed by cell tracing studies.

With respect to hematopoietic commitment, there is now compelling evidence from studies with the ES cell differentiation model and on the mouse embryo that the earliest identifiable progenitor is a cell that also displays vascular potential, a cell that is known as the hemangioblast (Choi et al., (1998); Development 125:725-732; Huber et al., (2004) Ngture 432:625-30). Analysis of this progenitor revealed that it co-expresses the mesoderm gene brachyury and the receptor tyrosine kinase Flk-1, indicating that it represents a subpopulation of mesoderm undergoing commitment to the hematopoietic and vascular lineages (Fehling et al., (2003) Development 130:4217-4227). Lineage-tracing studies have demonstrated that the heart develops from a Flk-1⁺ population, suggesting that a comparable multipotential cell may exist for the cardiovascular system (Ema et al., (2006) Blood 107:111-117). Analyses of ES cell differentiation cultures have provided evidence for the existence of a Flk-1⁺ progenitor with cardiac and endothelial potential (Yamashita et al., (2005) FASEB 19:1534-1536).

The Notch pathway is involved in cell fate determination and differentiation. The Notch pathway and Notch signaling are reviewed in Artavanis-Tsakanas (1995) Science 268:225-232. Four Notch proteins (Notch1, Notch2, Notch3 and Notch4) have been identified in humans. The Notch proteins are iransmembrane receptors. Upon activation by a ligand, the intracellular domain of Notch is proteolytically cleaved and transported to the nucleus to activate transcription of downstream effectors. Truncated forms of Notch that lack the extracellular ligand-binding domains are constitutively activated. See, e.g., U.S. Pat. No. 5,780,300.

Notch signaling is of interest in the context of early lineage commitment as it is involved in cell fate decisions in diverse developmental processes and it has been shown to play a role in hematopoietic, vasculogenic and cardiac development. The four different Notch receptors, Notch1-4, can associate with five ligands, delta-like 1-3 and jagged 1 and 2. Expression analyses of the early gastrulating mouse embryo revealed overlapping patterns for Notch1, 2, and 3 in the newly formed mesoderm. As gastrulation proceeds, distinct patterns emerge with Notch1 expression extending to developing blood islands in addition to other mesoderm subpopulations, while Notch1 expression overlaps with that of Notch1 in the paraxial and lateral plate mesoderm. Notch3 is detected in the cardiogenic plate in addition to the lateral plate and splanchnic mesoderm. With the establishment of the hematopoietic and cardiovascular systems, further segregation of expression is observed. All four genes have been reported to be expressed at some level in various hematopoietic lineages (review, Radtke et al., (2004) Nat. Immunol. 5:247-253). Notch1 is expressed in immature hematopoietic progenitors (Milner et al., (1994) Blood 83:2057-2062) as well as in the developing T cell lineage (Ellison et al., (1991) Cell 66:649-661). Within the vasculature, Notch1 is readily detected in endothelial and vascular smooth muscle cells (Loomes et al., (2002) Am. J. Med. Genet. 112:181-189), whereas Notch3 appears to be restricted to the smooth muscle lineage (Leimeister et al., (2000) Mech. Der. 98:175-178). Notch4 is found predominantly in the endothelial lineage (Uyttendaele et al., (1996) Development 122:2231-2239).

Despite these early and relatively broad expression patterns, targeting studies have demonstrated that the Notch receptors are not essential for gastrulation, germ layer induction or specification. Notch1 is essential for establishment of the definitive hematopoietic system as demonstrated by the failure of Notch1 mutant ES cells to contribute to definitive hematopoiesis in chimeric mice following injection into wild-type blastocysts (Hadland et al., (2004) Blood 104:2097-3105) and by the lack of hematopoietic development in Notch1⁺ AGM explants (Kumano et al., (2003) Immunity 18:699-711). Notch1 is also required for proper vascular morphogenesis as homozygous null embryos die at E11.5 from defects in angiogenic vascular remodeling (Krebs et al., (2000) Genes Dev. 14:1343-1352). In contrast to Notch1 mutants, Notch4 null animals are viable indicating that this receptor is not essential for embryonic development. Double mutant mice lacking both Notch1 and Notch4 display a more severe phenotype than Notch1 null embryos, demonstrating that Notch4 does play a role in development of a functional vascular system. Id. Notch2 is required for fetal development as the mutant embryos die between day 9.5 and 11.5 of gestation displaying extensive cell death in many tissues (McCright et al., (2001) Development 128:491-502) whereas Notch3 null mice are viable but do show some arterial defects (Domenga et al., (2004) Genes Dev. 18:2730-2735). The relatively late and variable defects observed in the Notch deficient animals despite the early expression patterns of their corresponding genes suggests that either this pathway is not essential during gastrulation or compensatory mechanisms could be masking the true function of some of the receptors.

Further insights into the role of notch signaling in hematopoietic, vascular and cardiac lineage commitment have come from forced expression studies in different model systems and in specific cell lines. The findings from such studies have demonstrated that Notch1 plays a critical role in the establishment of the γ/δ and α/β T cell lineages in the mouse (Washburn et al:, (1997) Cell 88:833-843) and that constitutive signaling through the receptor in early hematopoietic progenitors appears to favor their proliferation over differentiation, resulting in the emergence of immortalized progenitors with either lymphoid or myeloid characteristics (Varnum-Finney et al., (2000) Nat. Med. 6:1278-1281). In Zebrafish, Notch activation led to the expansion of hematopoietic cells in the AGM region during embryogenesis and enhanced hematopoietic recovery following radiation injury in the adult (Burns et al., (2005) Genes Dev. 19:2331-2342). While Notch signaling at appropriate stages enhances hematopoietic development, it appears to have an opposite effect on establishment of the cardiomyocyte lineage, as activation of Notch1 in the heart field of the Xenopus embryo was found to decrease the expression of cardiac markers (Rones et al., (2000) Development 127:3865-3876). Consistent with this finding is the observation that ES cells deficient in RBP-Jₖ, a downstream effector of the Notch pathway, appear to generate more cardiomyocytes than wild type counter parts while those expressing a constitutively active Notch1 receptor generated fewer (Schroeder et al., (2003) Prac. Natl. Acad. Sci. 100:4018-4023). The inhibitory effect of Notch signaling on cardiac development was demonstrated in the developing mouse as expression of the intracellular domain of the receptor repressed atrioventricular myocardial differentiation and ventricular maturation (Watanabe et al., (2006) Development 133:1625-1634). The effects of altered notch expression on the endothelial lineage are difficult to interpret as constitutive expression of Notch4 in endothelial cells in culture (Leong et al., (2002) Mol. Cell Biol. 22:2830-2841) or in the endothelial lineage of embryos (Uyttendaele et al., (2001) Proc. Natl. Acad. Sci. 98:5643-5648) inhibited endothelial sprouting and branching morphogenesis, whereas expression in a brain endothelial boll line induced the formation of microvessel like structures (Uyttendaele et al., (2000) Microvasc. Res. 60:91-103). Collectively, these findings indicate that Notch signaling can impact hematopoietic, vascular and cardiac development and that the observed effects are both stage and context specific.

It has been surprisingly discovered in accordance with the present invention that Notch signalling is involved in the specification of mesoderm to derivative lineages.

### SUMMARY OF THE INVENTION

The present invention provides cell populations that are enriched for cardiac progenitor cells and methods of making such cell populations.

The present invention thrther provides a method for inducing the differentiation of cardiac progenitor cells from embryonic stem (ES) cells comprising culturing ES cells under conditions sufficient to form EBs, culturing EBs under conditions sufficient for differentiation to hemangioblast/pre-erythroid cells, and isolating such cells and reaggregating in the presence of Notch.

The invention also provides a method for inhibiting the differentiation of cardiac cells from ES cells comprising culturing ES cells under conditions sufficient to form EBs, culturing the EBs under conditions sufficient for differentiation to a Bry⁺/Flk-1⁻ population, and isolating such a population and reaggregating in the presence of Notch.

The invention also provides a method of screening for an agent that has an effect on cardiomyocytes.

In another embodiment, the present invention provides a method of cardiomyocyte replacement therapy.

The methods of the present invention are useful for the expansion of precursor cells and for the generation of differentiated cells and tissues for cell replacement therapies, and for screening and identification of agents that affect cardiac progenitor cells and endothelial cells.

### BRIEF DESCRIPTION OF THE DRAWINGS

FIGS. 1A-D depict gene expression patterns of Notch4. FIG. 1A shows flow cytometric analysis of a day 3.25 populations demonstrating the GFP-Bry⁺/Flk-1⁺ hemangioblast and the GFP-Bry⁺/Flk-1⁻ cardiogenic populations. FIG. 1B shows expression of Notch4 in the GFP-Bry⁺/Flk-1⁺ and GFP-Bry⁺/Flk-1⁻ populations isolated from different staged EBs. FIG. 1C shows expression analysis of blast colony-derived core and outer cell populations. Four-day old blast colonies were picked from the methylcellulose cultures and the outer cells and cores were separated using a fine mouth pipette. Each population from individual colonies was analyzed for expression of the indicated genes. L32 was used was used as an internal control. FIG. 1D shows expression of Notch4 in ES cell-derived hematopoietic, endothelial and vascular smooth muscle cell lines. The HOX11-immortalized hematopoietic cell line EBHX11 and the endothelial cell line D4T (endo) were used for this analysis. The VSM cell line was established by force passaging EB-derived Flk-1⁺ cells. Expression of the indicated genes was used to verify the lineage fidelity of the 3 cell lines.

FIGS. 2A-D depict the effect of Notch4 signaling on hematopoietic development from the EB-derived F1k-1⁺ population. FIG. 2A shows expression of HA-Notch4 in ES cells 24 hours following Dox induction. For studies on the role of Notch4 signaling on hematopoietic development, day 3.25 Flk-1+ cells were isolated by cell sorting and reaggregated in serum-containing medium in the presence (+Dox) or absence (−Dox) of Dox (1 μg/ml) for 2 days. Following the Dox induction, the aggregates were dissociated and analyzed for hematopoietic potential. FIG. 2B shows flow cytometric analyses showing the proportion of VE-cad and CD41 positive cells in the aggregates. FIG. 2C shows the hematopoietic colony forming potential of the aggregate cells. Bars represent the standard error of the mean of the number of colonies from 3 cultures. Ep, primitive erythroid; Ed, definitive erythroid; Mac, macrophage; E/Mac, bipotential erythroid/macrophage. FIG. 2D shows gene expression analyses of aggregates.

FIGS. 3D-E depict the cardiac potential of the Notch4 induced Flk-1⁺ population. FIG. 3A shows the proportion of aggregates containing contacting cardiomyocytes following 24 hours of Dox induction of the day 3.25 Flk-1⁺ population. Single aggregates were plated into microtiter wells in the cardiac cultures and the presence of contracting cells was evaluated at 3 days following replating. −Dox/−Dox: uninduced cells, +Dox/−Dox: addition of Dox to the aggregation culture, +Dox/+Dox: addition of Dox to both of the aggregation and cardiac cultures, +Dox+inhibitor/−Dox: addition of Dox (0.5 μg/ml) and γ-secretase inhibitor (5 μM) to the aggregation culture. FIG. 3B shows immunostaining demonstrating the presence of cardiac Troponin T (cTnT) in cells from the induced (+Dox/−Dox) but not from the un-induced (−Dox/−Dox) aggregates. FIG. 3C is a flow cytometric analysis demonstrating the proportion cTnT⁺ cells present in cultures generated from pooled aggregates. Pools of aggregates were replated in the cardiac cultures for 3 days, at which time the cells were harvested and subjected to intracellular staining with an antibody to cTnT. The dark line represents cTnT⁺ cells whereas the shaded area represents control staining with secondary antibody alone. FIG. 3D shows gene expression analyses of the cardiac cultures 3 days following replating of the aggregates. Treatments are indicated on the top of the figure. FIG. 3E shows the proportion of cTnT positive cells that develop following removal of Dox from the cardiac cultures (+Dox/+Dox/−Dox).

FIGS. 4A and B depict the temporal developmental of the Flk-1⁺ EB population susceptible to cardiac induction by Notch4. Flk-1+ cells were isolated from day 3, 4 and 5 EBs and aggregated for 24 hours in the presence or absence of Dox. Aggregates from both groups were plated into microtiter wells and monitored for the development of contracting cells or subjected to gene expression analysis. Aggregates were monitored daily between 3 and 5 days of culture for the presence of contracting cells. FIG. 4A shows the proportion of aggregates that contained contracting cells. FIG. 4B shows the expression of nkx2.5 in the induced (+) and un-induced (−) aggregates from the different populations.

FIGS. 5A-F depict the effect of Notch4 expression on BL-CFC-derived blast colony development. Day 3.25 Flk-1⁺ cells were cultured in the raethylcellulose blast colony assay in the presence or absence of Dox. FIG. 5A is a photograph of blast (upper, −Dox) and compact (lower, +Dox) colonies following 4 days of culture. Original magnification 400×. FIG. 5B shows the number of blast or compact colonies generated in the absence or presence of Dox or in the presence of Dox and γ-secretase inhibitor. Colonies were scored following 4 days of culture. FIG. 5C shows gene expression analysis of individual compact and blast colonies. Each lane represents a single 7-day old colony. FIG. 5D shows immunostaining demonstrating the presence of cTnT in the adherent outgrowth of a single compact colony. The cells were grown on a glass coverslip for 4 days from a 7 day old compact colony. FIG. 5E is a photograph of a mixed lineage hematopoietic and cardiac colony (Original magnification 200×). Day 3.25 Flk-1⁺ cells were cultured for 1 day in the blast colony assay in the presence of Dox. Following this induction step, the entire contents of the methylcellulose culture was harvested, the developing colonies washed several times, and replated in the same volume in the blast colony assay in the absence of Dox. The secondary cultures were supplemented with Epo and IL-3 to enable visualization of erythropoiesis within the colonies. FIG. 5F shows gene expression analysis of individual mixed lineage colonies. Each lane represents a single 7 day-old colony.

FIGS. 6A and B depict induction of cardiac development in Flk-1⁺ population by the Notch ligand D11-1. Day 3.25 Flk-1⁺ cells from the Bry-GFP ES cell line were cultured on D11-1 expressing OP9 cells in serum-free conditions for 3 days, in the absence or presence of γ-secretase inhibitor (5 μM). Following this culture step, the cells were harvested, stained with the anti-cTnT antibody and analyzed by flow cytometry. FIG. 6A shows cells cultured in the absence of inhibitor. FIG. 6B shows cells cultured in the presence of inhibitor. The dark line represents cells stained with cTnT antibody whereas the shaded area represents control staining with secondary antibody alone.

FIGS. 7A-D show the role of Notch signaling on cardiac development from EB-derived GFP-Bry⁺/Flk-1⁻ mesoderm. Day 3.25 GFP-Bry⁺/Flk-1⁻ cells generated from the GFP-Bry/Ainv-Notch4·ES cell line isolated by FACS were reaggregated for 24 hours in the presence or absence of Dox or γ-secretase inhibitor. Following the reaggregation step, pools of aggregates were plated for 3-4 days in the cardiac cultures in the presence or absence of Dox or γ-secretase inhibitor. Populations cultured under the various conditions were analyzed for the presence cTnT⁺ cells by flow cytometry. FIG. 7A shows the proportion of cTnT⁺ cells that developed in the absence γ-secretase inhibitor (−I/−I), or from cells exposed to γ-secretase inhibitor during the reaggregation step (+I/−I) or in the cardiac cultures (−I/+I). FIG. 7B shows cardiac gene expression of the cells grown in the cardiac cultures in the presence or absence of γ-secretase inhibitor. FIG. 7C shows the proportion of cTnT⁺ cells that develop in the absence or presence of Dox induction. (−Dox/−Dox), non-induced cells; (+Dox/−Dox), Dox added during the reaggregation step; (−Dox/+Dox) Dox added to the cardiac cultures. FIG. 7D shows cardiac gene expression of the cells cultured in the presence or absence of Dox.

FIGS. 8A-D depict the role of Notch signaling in cardiac development from E7.5 primitive streak explants. FIG. 8A is a photograph of an E7.5 embryo indicating the dissection scheme used to generate the distal primitive streak (DPS), and the posterior primitive streak (PPS) for Notch gene analyses. FIG. 8B shows expression analyses of the PPS and DPS. FIG. 8C shows the percentage of PPS explants that had contracting cells after 5 days of culture in the presence (+inhibitor) or absence (−inhibitor) of γ-secretase inhibitor. FIG. 8D shows gene expression analyses of the PPS explants cultured for 5 days in the presence or absence of γ-secretase inhibitor.

### DETAILED DESCRIPTION OF THE INVENTION

In accordance with the present invention, it has been discovered that the differentiation of ES cells can be directed by activating or inhibiting Notch in ES-derived cells or their progeny. Notch is defined herein to include Notch1, Notch2, Notch3, Notch4 and active variants and fragments thereof, including active truncated forms that lack the extracellular ligand-binding domain. The terms activation and inhibition of Notch as used herein refer to activation and inhibition of the Notch signaling pathway. Accordingly, activation of Notch may be accomplished by contacting a cell with a Notch agonist including for example a Notch ligand, or introducing into a cell a recombinant nucleic acid that expresses activated Notch or another molecule that activates the Notch pathway. Notch agonists are known in the art and include, for example, the Notch ligands Delta-like1-3 and Jagged1 and 2. Inhibition of Notch may be accomplished by contacting a cell with a Notch antagonist or introducing into a cell a recombinant nucleic acid that inhibits Notch or inhibits the Notch pathway. Antagonists are known in the art and disclosed for example by Dontu et al. (2004) Breast Cancer Res. 6:R.605-R615.

A nucleic acid that expresses Notch or another molecule that activates the Notch pathway, or that inhibits Notch or the Notch pathway may be introduced into an ES cell or an ES-derived cell by methods known to those of ordinary skill in the art, including gene transfer by viral vectors, homologous recombination, and recombinase-based approaches. In a preferred embodiment, the nucleic acid is operably linked to a regulatory element that controls inducible expression such that expression of a nucleic acid that activates or inhibits Notch is inducible. In a most preferred embodiment, a doxycycline inducible (“dox-on”) gene expression system is. used. Such systems are known in the art and disclosed for example by Ting et al. (2005) Methods in Molecular Medicine 105:23-46.

In a preferred embodiment, a recombinant nucleic acid that expresses activated Notch is introduced into a cell. In another preferred embodiment, the recombinant nucleic acid encodes Notch4 or an active fragment thereof. The nucleic acid sequences of human and mouse Notch4 are known. Uyttendaele et al. (1996) Development 122:2251-2259; Li et al. (1998) Genomics 51:45-58. In a preferred embodiment, the nucleic acid encodes the constitutively active intracellular domain of Notch4. Thus truncated form of Notch4 (Notch4-IC) is disclosed in the art, for example by Soriano et al. (2000) International Journal of Cancer 86:652-659 and Vercauteren et al. (2004) Blood 104:2315-2322. In a preferred embodiment, the nucleic acid has a sequence that encodes amino acids 1476-2003 of human Notch4 (as numbered by Li et al., supra). In another embodiment, the nucleic acid has a sequence that is at least 80%, or preferably at least 90%, or more preferably at least 95% homologous to the sequence that encodes amino acids 1476-2003 of human Notch4.

ES cells may be obtained commercially or by methods known in the art. For example, ES cells may be obtained from blastocysts by methods known in the art and disclosed for example by Evans et al. (1981) Nature 292:154-156; Thomson et al. (1995) Proc. Nat'l. Acad. Sci, USA 92:7844; U.S. Pat. No. 5,843,780; and Reubinoff et al. (2000) Nature Biotech. 18:399. In a preferred embodiment the ES cells are mouse or primate ES cells. In another preferred embodiment, the ES cells are human ES cells.

In one preferred embodiment, ES cells may be engineered to inducibly express the active intracellular domain of Notch4 by the methods described above and for convenience are referred to herein as “Notch4-ES cells.” Such ES cells and their progeny express activated Notch4 upon exposure to the appropriate inducer. In a preferred embodiment the expression system is a dox-on system inducible by doxycycline.

Thus in one embodiment, the present invention provides a method of inducing differentiation of cardiac progenitor cells from ES cells comprising culturing ES cells for a time and under conditions sufficient for formation of embryoid bodies (EBs), culturing the EBs for a time and under conditions sufficient for differentiation to hemangioblast/pre-erythroid cells, and isolating and reaggregating the hemangioblast/pre-erytbroid cells in the presence of activated Notch to provide cardiac progenitor cells. The cardiac progenitor cells may be cultured under conditions sufficient for differentiation to cardiomyocytes. In another embodiment, the method further comprises the step of culturing the cardiac progenitor or cardiomyocytes cells under cardiac culture conditions in the absence of activated Notch.

EBs are three dimensional colonies that contain developing populations from a broad spectrum of lineages. Conditions for formation of BBs are known in the art and disclosed for example by Smith (2001) Annu. Rev. Cell Dev. Biol, 17:435462 and WO 2004/098490 to Keller et al. As a nonlimiting example, ES may be cultured in Iscove Modified Dulbecco Medium (IMDM) supplemented with 2 mM L-glutamine, 200 μg/mL transferrin, 0.5 mM ascorbic acid, 4×10⁻⁴M monothioglycerol plus 15% fetal calf serum to generate EBs. EBs may be cultured in the presence of serum for a time sufficient for differentiation to a hemangioblast/pre-erythroid population. In a preferred embodiment the BBs are cultured for about 2.5 to 4.5 days. In a more preferred embodiment, ES cells are cultured for about 3 days. Hemangioblast/pre-erythroid cells are deftned herein as Bry⁺/Flk-1⁺ and are collected, for example by sorting and isolating cells expressing a marker indicative of these cells such as the tyrosine kinase receptor VEGRF2 also known as KDR or Flk-1. Methods for sorting of KDR⁺ and Flk-1⁺ cells are known in the art and disclosed for example by WO 2004/098490 to Keller et al.

To induce cardiomyooyte differentiation, the hemangioblast/pre-erythroid cells are reaggrogated under conditions whereby Notch is activated. In a preferred embodiment, serum free conditions are used. In another preferred embodiment, Notch is activated for about 12-48 hours. In a more preferred embodiment, Notch is activated for about 24 hours. Notch may be activated, as described hereinabove, e.g., by adding a Notch agonist or by inducing expression of a nucleic acid encoding Notch that has been introduced into the ES cell. For example, if doxycycline-inducible Notch4-ES cells are used, doxycycline is added for about 12-48 hours, and preferably for about 24 hours. Single aggregates may then be picked and cultured under cardiac differentiation conditions in the absence of activated Notch. Such conditions are known in the art and include, for example, culturing in serum-free medium. Cardiomyocyte differentiation may be determined by monitoring for the development of beating cell masses, by assaying for the presence of a cardiac marker such as Troponin-T, or by detecting gene expression of cardiovascular markers such as Nkx 2.5.

The hemangioblast/pre-erythroid cells, in the absence of Notch activation, differentiate to the hematopoietic and vascular lineages. Accordingly, by the discovery that Notch activation redirects this population to cardiac cells, the present invention provides a novel source of such cells.

The foregoing method provides cell populations that contain at least about 10% cardiomyocytes. In a preferred embodiment the cell populations comprise at least about 50% cardiomyocytes. In more preferred embodiments, the cell populations comprise about 60%, or about 70%, or about 80%, or most preferably about 90% cardiomyocytes.

The cell populations enriched for cardiomyocytes are useful in a method for the screening for an agent that has an effect on cardiomyocytes. The method may be used, for example, to identify agents that alter lineage development improve cell function, alter differentiation to sublineages, affect contractile activity, or promote proliferation and maintenance of cells in long term culture. The method may be used for screening of pharmacological compounds for toxicity and efficacy. The method of screening for an agent that has an effect on cardiomyocytes comprises contacting cardiomyocytes of the present invention with a candidate agent and assaying for an effect on the cardiomyocytes in the presence of the agent, whereby the presence of an effect is indicative of the identification of an agent that has an effect on cardiomyocytes.

Examples of candidate agents include, but are not limited to, nucleic acids, carbohydrates, lipids, proteins, peptides, peptidomimetios, small molecules and antibodies. Candidate agents may be naturally occurring or synthetic, and may be obtained using combinatorial library methods.

The effect on cardiomyocytes may be determined by any standard assay for phenotype or activity, including for example an assay for marker expression, receptor binding, contractile activity, electrophysiology, cell viability, survival, morphology, or DNA synthesis or repair.

The cell populations enriched for cardiomyocytes are also useful for cell replacement therapies, and may be used for example for treatment of a disorder characterized by insufficient cardiac function including, for example, congenital heart disease, coronary heart disease, cardiomyopathy, endocarditis or total heart block. Accordingly, in one embodiment the present invention provides a method of cardiomyocyte replacement therapy comprising administering to a subject in need of such treatment a composition comprising cardiomyocytes isolated from a cell population enriched for cardiomyocytes obtained in accordance with the present invention. In a preferred embodiment, the subject is a human. The composition may be administered by a route that results in delivery to cardiac tissue including, for example, injection or implantation.

The present invention also provides a method of inhibiting the differentiation of cardiac cells from ES cells and ES-derived cells. The method comprises culturing ES cells for a time and under conditions sufficient for differentiation and formation of EBs, culturing the EBs for a time and under conditions sufficient for differentiation to a Bry⁺/Flk-1⁻ cell population, and isolating and reaggreating the Bry⁺/Flk-1⁻ cell population in the presence of an inhibitor of Notch under conditions whereby differentiation of cardiac cells is inhibited. Inhibition may be measured as described above, for example by detecting cell surface markers and lineage specific gene expression. Inhibitors of Notch4 are known in the art and include, for example, γ-secretase inhibitor X. In a preferred embodiment, EBs are cultured for about 2.5 to 4.5 days. In another preferred embodiment, EBs are cultured for about 3 days. In another preferred embodiment, the cells are reaggregated in the presence of the Notch inhibitor for about 24 hours. The method optionally comprises the further step of culturing single aggregates under cardiac culture conditions in the presence of an inhibitor of Notch.

All references cited herein are incorporated herein in their entirety.

The following examples serve to thither illustrate the present invention.

### EXAMPLE 1

### Materials and Methods

ES Cell Culture and Differentiation

ES cells were maintained on irradiated feeders in Dulbecco's Modified Eagle Medium (DMEM) supplemented with 15% fetal calf serum (FCS), 10% ES cell conditioned medium, penicillin, streptomycin, 1.5×10⁻⁴ M monothioglycerol (MTG; Sigma) and LIF (1% conditioned medium). Prior to induction of differentiation, cells were passaged 2 times on gelatin-coated plates in Iscove Modified Dulbecco Medium

(IMDM) containing the same supplements mentioned above to deplete the population of feeder cells. For the generation of BBs, the cells were harvested and cultured in 60 mm low attachment Petri grade dishes (VWR) with IMDM supplemented with 2 mM L-glutamine (Gibco/BRL), 200 μg/mL transferrin (Boehringer Mannheim.), 0.5 mM ascorbic acid (Sigma), 4×10⁻⁴ M MTG plus 15% FCS. For reaggregation cultures to support the differentiation of the heraatopoietic and vascular lineages, 3×10⁵ Flk-1⁺ cells/ml were cultured for 2 days in ultra-low attachment 24-well plates (Corning Costar) with the same EB differentiation medium plus 5% Protein-Free Hybridoma Medium-II (PFHM-II, Invitrogen).

Notch4 Inducible ES cells

The activated form of Notch4 cDNA (int-3) tagged with hemagglutinin (HA) sequence is described by Uyttendaele (1996) Development 122:2251-2259. The tet-on inducible ES cell line, Ainv18, described by Ting et al. (2005) Methods Mol. Med. 105:23-46, was further modified by targeting the EGFP cDNA into brachruy locus as described by Fehling et al., (2003) Development 130:4217-4227. The Notch4 cDNA was introduced into the Ainv 18 and the modified Ainv ES cell lines by the approach described by Kyba et al., (2002) Cell 109:29-37. Briefly, the cDNA fragment of the activated form of Notch4 tagged with HA was inserted to the plox plasmid by convenient restriction sites to generate plox-Notch4/HA. Ainv 18 and the modified cell line were targeted with plox-Notch4/HA by coelectroporation of 40 μg each of plox-Notch4/HA and the Cre recombinase expression plasmid, pSalk-Cre. Positive clones were screened in ES medium with 300 μg/ml G418 (GIBCO) and isolated to generate inducible cell lines, Ainv-Notch4 and GFP-Bry/Ainv-Notch4. The positive clones were confirmed by immunohistochemistry. detecting HA expression after induction.

### Flow Cytomery

Dissociated cells were incubated with biotinylated mAbs (against Flk-1, VE-cad, or CD41) in PBS containing 10% FCS on ice for 30 min. The cells were then washed once and incubated with streptavidin-PE-Cy5 (BD Pharmingen) for another 30 minutes on ice. Following an additional two washes, the cells were analyzed on a FACSCalibur flow cytometer (BectonDicldnson) or sorted on a Moflo cell sorter (Cytomation). For Troponin T or HA staining, cells were fixed in 4% paraformaldehyde (PFA) for 30 minutes and then incubated in a permeabilizing buffer consisting of PBS with 10% FCS and 0.1% saponin (Sigma) for 10 minutes. Following fixing and permeabilization, the cells were washed twice and incubated with an anti-Troponin T (unconjugated mouse antibody, Lab Vision) or anti-HA (conjugated with biotin, Covance) antibody for 30 minutes. After two washes, the cells were incubated with a secondary APC-conjugated goat anti-mouse antibody (for Troponin T antibody) or streptavidin-PE-Cy5 (for biotinylated HA antibody) for 30 minutes. Finally, the cells were washed twice with permeabilizing buffer and then twice with buffer without saponin.

### Colony Assays

The blast and hematopoietic colony assays were performed as described Kennedy et al., (2003) Metods Enzymol. 365:39-59. Dox was added at 0.5 μg/ml to induce Notch4 expression and y-secretase inhibitor X (L685,458, Calbiochem) at 5 μM to blook Notch signaling in the:blast colony culture. To generate mixed hemangioblastkardiac colonies, blast colony growth was initiated in standard blast colony cultures containing Dox for 24 hours. The developing colonies were then washed from with methycellulose with IMDM containing 10% FCS to remove Dox. The colonies were recultured in blast colony metycellulose supplemented with Erythropoietin (2 U/ml) and IL-3 (1% conditioned medium). Mixed colonies containing an inner cardiac core surrounded by outer hematopoietic cells were picked for analysis at day 7.

### Cardiac Assay

Sorted cells were reaggregated for 24 hours in StemPro-34 serum-free medium (hwitrogen) containing 2 mM L-glutamine (GIBCO/BRL), transferrin (200 μg/ml), 0.5 mM ascorbic acid and 4.5×10⁻⁴ M MTG at 3×10⁵ cells per ml in ultra-low-attachment 24-well plates (Costar). Single aggregates or pools of aggregates were replated in gelatin-coated 96- or 24-well plates containing SternPro with 2 mM L-glutamine for cardiac culture. Following 2 to 4 days of culture the proportion of aggregates containing contracting cells was scored and the number of Troponin T-positive cells was evaluated by flow cytometric analyses. For the aggregated and cardiac cultures, doxycycline (Dox) was used at 0.5 μg/ml and γ-secretase inhibitor X at 5 μM (dissolved in DMSO). The same concentration of DMSO was added to the control cultures. Medium was changed everyday to provide fresh Dox and inhibitor.

### Gene Expression Analysis

Gene expression analyses of colonies or small amount of mRNA was performed by polyA⁺ global amplification polymerase chain reaction (PCR) as described by Robertson et al., (2000) Development 127:2447-2459. Amplified PCR products were resolved on agarose gels and transferred to a Zeta-probe GT membrane (Bio-Rad). Genes of interest were then probed by ³²P randomly primed cDNA fragments (Ready-to-Go Labeling; Pharmacia) corresponding to the 3′ regions of the genes. For gene-specific PCR, total RNA was extracted from cells using the RNeasy mini-kit (Qiagen). One microgram total RNA was used to generate cDNAs by reverse transcription using the Omiliscript RT kit (Qiagen) with random hexamer and then the cDNAs were subjected to PCR.

### Immunohistochemistry

Cell aggregates or colonies were plated on gelatin-coated coverslips and cultured for 3 days in StemPro with 2 mM L-glutamine. Cells cultured on coverslips were fixed in 4% paraformaldehyde for 30 minutes, washed two times in PBS, permeabilized in 0.2% TritonX-100/PBS for 10 minutes, and washed in 10% FCS/1% Tween 20/PBS. Cells attachedto the coverslips were incubated for 1 hour with an antibody againstthe cardiac Troponin T. After 3 washes, the cells on ooverslips were incubated with FTTC-conjugated goat anti-mouse antibody (Jackson ImmunoResearch) for 1 hour in the dark. Finally, the coverslips were washed 3 times and then inverted onto a drop of DAPI (Vector Laboratories). Fluorescence was visualized using a Leica DMRA2 fluorescence microscope (Wetzlar).

### Cell Culture on D11-1 Expressing Stromal Cells

OP9-DL1 cells described by Schmitt et al. (2004) Nat. Immunol. 5:410-417 were cultured in a 24-well plate and irradiated before use. Day 3.25 EB-derived Flk-1⁺ cells (3×10⁴ per well) were seeded onto OP9 cells in the same medium used for the cardiac cultures. γ-Secretase inhibitor X (dissolved in DMSO) at 5 μM or a corresponding volume of DMSO was included in the cultures. Medium was changed everyday to supply fresh inhibitor. After 3 days of culture, the cells were harvested and subjected to flow oytometric analysis to determine the number of Troponin T-positive cells.

### Embryo Dissections and Explant Cultures

Female swiss webster mice (Taconic) were mated with male GFP-Bry^{+/-} mice described by Huber et al., (2004) Nature 432:625-630. Pregnant mice were sacrificed 7.5 days after mating and the embryos were isolated. Dissections were performed under a Leica MZFLIII fluorescence dissecting stereomicrosope to visualize the GFP expression in the primitive streak (PS). Using tungsten needles (Fine Science tools), the PS of GFP-Bry^{+/-} embryos were isolated and separated into posterior and anterior regions. Individual anterior and posterior PS pieces were plated in gelatin-coated 96-well dishes with medium for cardiac cultures. γ-Secretase inhibitor at 10 μM or a corresponding volume of DMSO was included in the cultures. Medium was changed everyday to provide fresh inhibitor. After 3-5 days, the explants were scored for the presence of contracting foci and harvested for gene expression analysis.

### EXAMPLE 2

### Notch Expression in ES Cell-Derived Populations

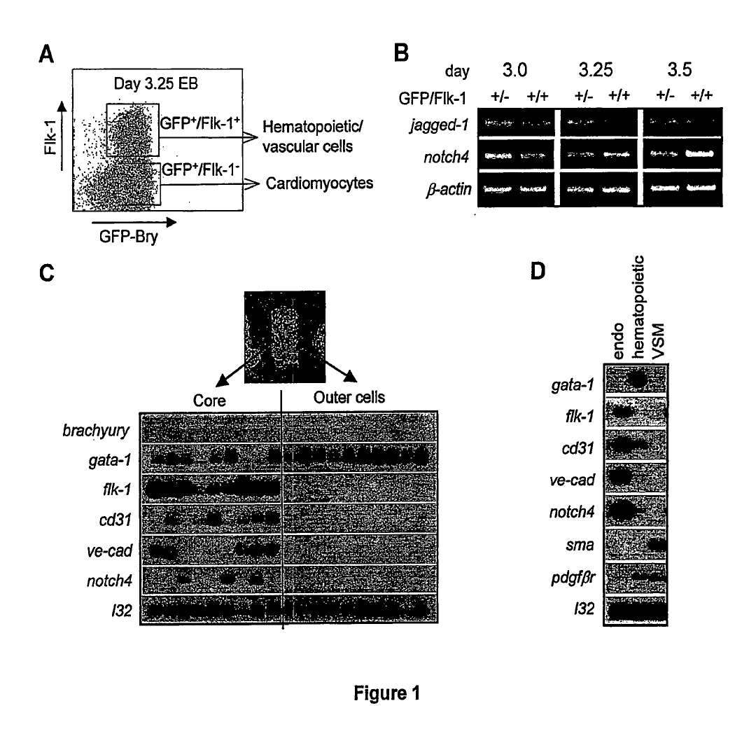

The expression of Notch4 was evaluated in early mesoderm populations that arise during embryoid body (EB) differentiation, focusing on some of the earliest cells during the commitment to cardiac, hematopoietic and vascular fates. Following 3.0-3.5 days of serum stimulation, ES cells with the green fluorescent protein (GFP) cDNA targeted to the brachyury locus (GFP-Bry) generate three distinct populations based on Flk-1 and GFP expression; GFP-Bry⁻/Flk-1⁻, GFP-Bry⁺/Flk-1⁻ and GFP-Bry⁺/Flk-1⁺ (FIG. 1A). Functional studies have shown that the GFP-Bry⁺/Flk-1⁺ population at early stages of differentiation contains hemangioblasts whereas the GFP-Bry⁺/Flk-1⁻ population displays cardiac potential (Kouskoff et al., (2005) Proc. Natl. Acad. Sci. 102:13170-13157). Expression analysis revealed that Notch4 was expressed in both the GFP-Bry⁺/Flk-1⁻ and GFP-Bry⁺/Flk-1⁺ populations, isolated at days 3.0, 3.25 and 3.5 of differentiation. The relative expression levels appear to shift between these populations over this time frame, with higher Notch4 levels being detected in the GFP-Bry⁺/Flk-1⁻ cells at day 3.0 and in the GFP-Bry⁺/Flk-1⁺ cells at day 3.5 (FIG. 1B). Expression of jagged-1, a Notch ligand, was detected in both populations, although the levels appeared to be higher in the GFP-Bry⁺/Flk-1⁻ population at the two later time points. Notch1, 2 and 3 were also expressed in both populations at these times.

When the GFP-Bry⁺/Flk-1⁺ population is plated in methylcellulose cultures in the presence of VEGF and IL-6, these cells generate blast colonies that display both hematopoietic and vascular potential (Fehling et al., (2003) Development. 130:4217-4227). The progenitor that gives rise to these colonies, the blast colony-forming cell (BL-CFC), is considered to represent the in vitro equivalent of the hemangioblast. When analyzed early in their development two morphologically distinct populations can be detected in these colonies, an inner core surrounded by an outer population (FIG. 1C). These populations were separated by pipetting and subjected to expression analysis by PCR. The outer cells expressed gata-1, but none of the endothelial genes, indicating that they represent developing hematopoietic cells. The core samples expressed the endothelial genes as well as low levels of gata-1, suggesting that they consist of a mixture of hematopoietic and endothelial cells. Notch4 expression was restricted to the core populations. In addition to the blast colonies, the expression of Notch4 was also analyzed in three ES cell-derived cell lines, representing the endothelial, hematopoietic and vascular smooth muscle lineages (FIG. 1D). Notch4 was only detected in the endothelial cell line, confirming its endothelial-restricted pattern. Taken together, these observations indicate that Notch4 as well as the other Notch genes are expressed broadly in mesodermal populations at early stages of ES cell differentiation. Expression of Notch4 becomes restricted to the endothelial lineage following hemangioblast specification.

### EXAMPLE 3

### Forced Expression of Constitutively Activated Notch4 in the Hemangiobiast-Containing Flk-1⁺ Population Inhibits Hematopoietic Differentiation

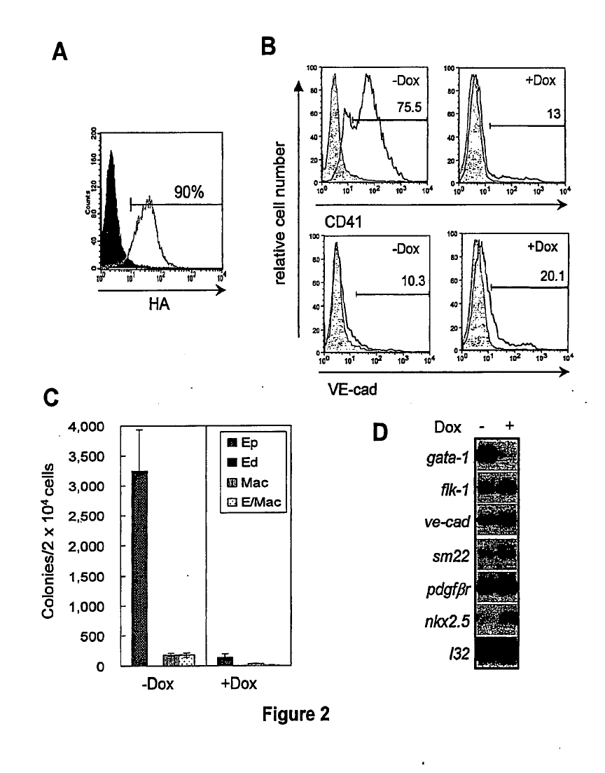

To determine if Notch4 plays a role during hematopoietic and vascular commitment, an inducible ES cell line that expresses an active form of Notch4 was generated. A cDNA encoding the intracellular domain of Notch4 (Notch4-IC), was engineered into the Ainv18 ES cells. This form of the receptor contains the anchored domain that requires cleavage by the ubiquitous enzyme γ-secretase for activation. With the Ainv ES cell system, expression of the gene of interest is induced by tetracycline or its analog, doxycycline (Dox). A hemagglutinin epitope (HA) sequence was inserted at the carboxyl terminus of the Notch4 cDNA to enable detection of the expressed protein. The Ainv-Notch4 ES cell line displayed identical differentiation kinetics to the parental Ainv18 line with respect to expression patterns of markers indicative of endothelial (Flk-1, VE-cad) and hematopoietic (CD41) development. One day following Dox (0.5 μg/ml) induction, 90% of Ainv-Notch4 ES cells expressed Notch4 as determined by flow cytometric analysis for HA expression (FIG. 2A).

To investigate the effects of Notch4 signaling on the specification of the hematopoietic and endothelial lineages, this pathway was induccid in a population of EB-derived cells undergoing hemangioblast development. The hemangioblast stage of differentiation, as defined by the presence of the BL-CFC, is found in the Flk-1⁺ population between days 2.75 and 4.0 of EB development for most ES cell lines. Flk-1⁺ cells isolated from day 3.25 EBs by fluorescent activated cell sorting (FACS) were cultured at high cell density in serum-containing differentiation medium for 2 days to form aggregates that support the differentiation of the BL-CFC to the hematopoietic and vascular lineages. In the absence of Dox, the Flk-1⁺ population generated a large CD41⁺ hematopoietic population (FIG. 2B) and large numbers of hematopoietic progenitors (FIG. 2C) during the 2-day reaggregation step. Addition of Dox dramatically reduced the size of the CD41⁺ population and the hematopoietic progenitor content of the aggregates, indicating that Notch4 inhibited hematopoietic development from this F1k-1 population. Induction of Notch4 resulted in a small increase in the proportion of VE-cad⁺ endothelial cells in the aggregates (FIG. 2B). Gene expression profiles confirmed the inhibitory effects of Notch4 overexpression on hematopoietic development. Aggregates from the induced cultures expressed considerably lower levels of the hematopoietic specific gene gata-1 compared to the aggregates from non-induced cultures (FIGS. 2D). In contrast, expression of genes indicative of endothelial and vascular smooth muscle development including, flk-1, ye-cad, SM22 and pdgifir, were up-regulated in the Notch4-induced aggregates (FIG. 2D). Induction of Notch4 also led to the expression of Nkx2.5, a gene normally expressed during the early stages of cardiac specification . This example demonstrates that Notch4 over-expression in Flk1+ cells from day 3 EBs inhibits hematopoietic differentiation.

### EXAMPLE 4

### Notch4 Over-Expression Redirects the Fate of Non-Cardiogenic Flk-1⁺ Cells to Cardiomyocytes

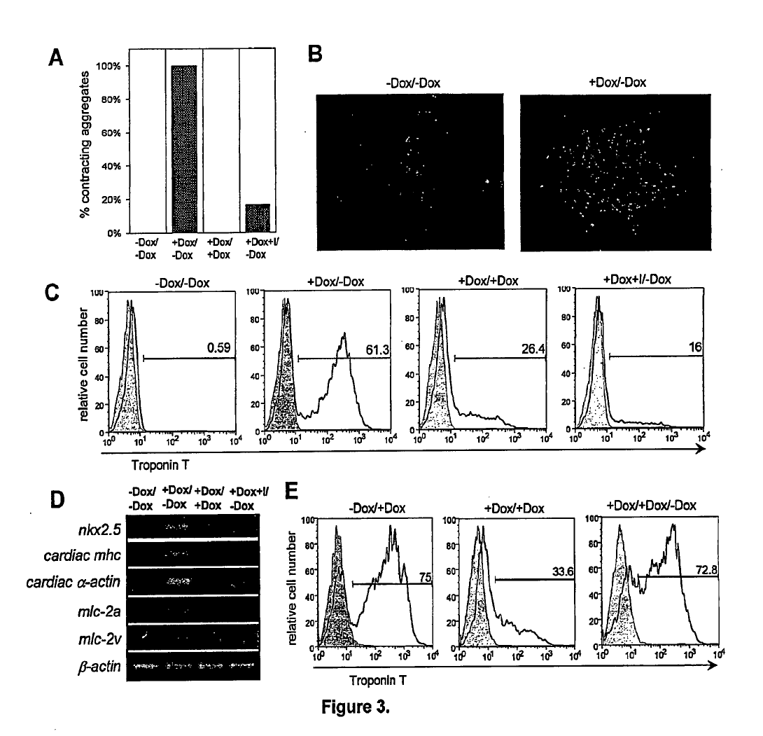

To investigate the potential of Notch4 to initiate a cardiogenic program in this early stage of hemangioblast population, Flk-1⁺ cells isolated from day 3.25 Ainv-Notch4 EBs were reaggregated for 24 hours in the presence or absence of Dox as described above. The resulting aggregates were then cultured in gelatin-coated microliter wells containing serum-free media (hereafter referred to as cardiac cultures). These conditions efficiently support cardiomyooyte development from cardiogenic mesoderm (Kouskoff et al. (2005) Proc. Natl. Acad. Sci. 102:13170-13175). Both single aggregates and pools of aggregates were cultured for 2-3 days. Following this maturation step, the cultures of single aggregates were scored for the presence of contracting cells indicative of cardiomyocyte differentiation. None of the aggregates generated in the absence of Dox (−Dox/−Dox) contained contracting cells (FIG. 3A). Rather, these aggregates underwent heniatopoietio differentiation as indicated by the development of hemoglobinized erythroid cells, an observation consistent with the hemangioblast potential of this population. In contrast, all aggregates from the population induced for 24 hours contained contracting cells (+Dox/−Dox, FIG. 3A). Immunostaining of the contracting cells from individual aggregates demonstrated the presence of the cardiac form of Troponin T (cTnT) further confirming the cardiomyocyte nature of these cells. (FIG. 3B, right panel). Few cTnT cells were atected among the adhesive cells generated from non-induced aggregates (FIG. 3B, left panel). Cultures of the pooled induced aggregates generated extensive areas of contracting cells. Contracting cells were not detected in the cultures of the non-induced aggregates. Flow cytometric analysis of the differentiated progeny from pooled inducedaggregates confirmed the dramatic cardiogenic effect of Notch4 as greater than 60% of the entire cell population expressed cTnT after 2 days in the cardiac cultures (+Dox/−Dox, FIG. 3C). Less than 1% of the cells generated from the non-induced population expressed cTnT (−Dox/−Dox, FIG. 3C). Consistent with the cTnT expression and the presence of contracting cells, the induced populations expressed cardiac specific genes including nkx2.5, cardiac mhc, α-actin, mlc2a and mlc2v (FIG. 3D). The generation of contracting cells and expression of cardiac genes in the aggregate-derived populations could be inhibited by blocking Notch4 signalling with γ-secretase inhibitor during the reaggregation step (+Dox+inhibitor/−Dox, FIG. 3A, 3C and 3D). This reversal of fate by the inhibitor is a clear demonstration that the observed induction of the cardiac lineage is dependent on Notch signalling.

If Dox was maintained during the plating of the aggregates in the cardiac cultures, no contracting aggregates.were observed, the cTnT-positive population was significantly reduced in size and the expression of the cardiac genes down regulated (+Dox/+Dox, FIG. 3 A, 3C and 3D). To determine if cardiac potential was maintained in these cultures, Dox was removed following 2 days of exposure in the cardiac cultures and the cells were grown for an additional 2 days in the absence of Dox. As shown in FIG. 3E, a large population of cTnT-expressing cells developed in these cultures within 2 days of Dox removal (Dox+/Dox+/Dox−, FIG. 3E). Populations of contracting cells were readily detected in these cultures. These observations indicate that prolonged expression of Notch4 inhibited maturation of the cardiac lineage. Maturation did progress following the removal of Dox, indicating that cardiac potential did persitt in the population. This example demonstrates that activation of Notch4 signalling is able to redirect the fate of the early non-cardiogenic Flk-1⁺ cells to cardiomyocytes at the expense of hematopoietic progenitor cells and that the duration of the Notch4 induction affects the cardiac fate determination.

### EXAMPLE 5

The Cardiogenic Effect of Notch4 is Restricted to the Flk-1⁺ Cells from Early Stage, - EBs

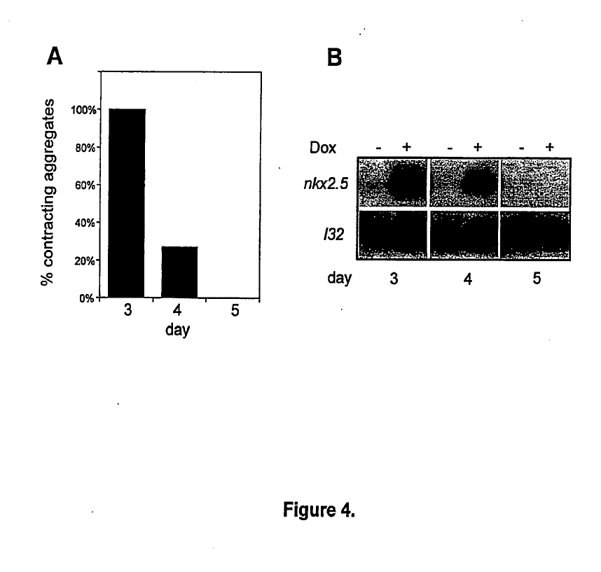

BL-CFCs are found in the Flk-1⁺ population between days 2.75 and 4 of EB differentiation. Beyond this stage, this population consists of restricted hematopoietic and vascular progenitors. To determine if the cardiogenic effect of Notch4 was restricted to the hemangioblast stage or if it could be observed in later stage Flk-1 populations, Flk-1⁺ cells were isolated from day 3, 4 and 5 EBs, reaggregated in the presence or absence of Dox and then evaluated for cardiac potential. All aggregates from the day 3 Flk-1⁺ cells contained contracting cells (FIG. 4A). In contrast, only 25% of aggregates from the day 4 Flk-1⁺ cells and none from the day 5 population displayed this activity. Analysis of nkx2,5 expression immediately following the aggregation step demonstrated the presence of the transcripts in the aggregates from the day 3 and 4 Flk-1⁺ cells but not in those from the day 5 Flk-1⁺ cells (FIG. 4B), an observation consistent with the distribution of contracting cells. The findings from this kinetic analysis demonstrate that the effects of Notch4 are stage specific and indicate that the population that can undergo fate change is transient and restricted to the hemangioblast stage Flk-1⁺ cells.

### EXAMPLE 6

Notch4 Induction Switches the Potential of the BL-CFC from a Hematopoietic to a Cardiac Fate.

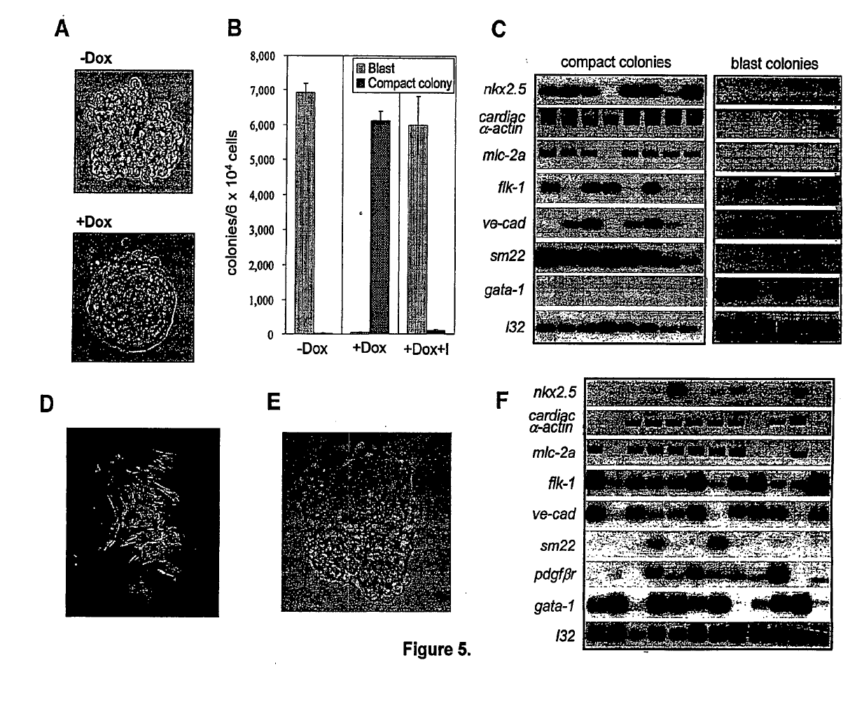

To determine if the BL-CFC is the target of the Notch4-induced fate change, day 3.25 Flk-1⁺ cells were cultured in the BL-CFC assay in the presence or absence of Dox. In the absence of Dox, this population generated typical blast colonies that appeared as grape-like clusters of cells. When cultured in the presence of Dox, these cells formed compact colonies of tightly packed cells that were easy to distinguish from the blast colonies (FIG. 5A). The number of these compact colonies was similar to the number of blast cell colonies that developed in the non-induced cultures (FIG. 5B). Addition of γ-secretase inhibitor together with Dox resulted in a reversal back to blast colonies, indicating that the development of the compact colonies was mediated by Notch signalling. Molecular analysis revealed that most of the compact colonies expressed the cardiac genes nkx2.5, cardiac α-actin and mlc-2a, the endothelial genes flk-1 and ve-cad and the vascular smooth muscle gene sm22 (FIG. 5C, left panel). None of these colonies expressed gata-1. As shown previously, blast colonies expressed the endothelial genes as well as gata-1. They did not express appreciable levels of the cardiac genes (FIG. 5C, right panel). With extended time in the methylcellulose cultures, some of the compact colonies generated contracting cells. To quantify the proportion of colonies that generated contracting cells, individual colonies were picked at day 7 of culture and replated in microtiter wells in the cardiac cultures. Approximately 70% of the compact colonies generated contracting cells between 2 and 7 days of culture. The contracting cells expressed cTnT, confirming that they were cardiomyocytes (FIG. 5D). Blast colonies did not give rise to contracting cells when grown in the cardiac cultures. The expression profile and developmental potential of the compact colonies suggests that they represent colonies of vascular and cardiac cells.

The appearance of the compact colonies in place of the blast cell colonies following Dox induction could be due to the fact that expression of Notch4 induced the growth of a novel progenitor while inhibiting the development of the BL-CFC. Alternatively, expression of Notch4 in the BL-CFC may redirect its fate from the hematopoietic to the cardiac lineage. The observation that comparable numbers of blast and compact colonies developed is consistent with the latter interpretation. To further investigate the origin of the compact colonies, the exposure of the BL-CFC to Dox was limited to 24 hours. At this stage, the developing colonies were removed from the Dox-containing methylcellulose and repleted in hemangioblast methylcellulose supplemented with Epo and IL-3 to promote the expansion of any hematopoietic cells. If Notch4 was acting on the BL-CFC, a restricted induction period might initiate cardiac development without completely inhibiting hematopoiesis, resulting in the development of mixed hernatopoietickardiac colonies. Following 5 days of culture, colonies containing an inner core of cells surrounded by hematopoietic cells could be observed (FIG. 5E). Some of the cores began contracting after 7 days of the methylcellulose cultures. When picked and replated into the cardiac cultures in microliter wells, 45% of these mixed colonies generated contracting cells. Molecular analysis of these colonies confirmed the presence of the hematopoietic (gata-1), endothelial (flk-1 , ve-cad) and cardiac (cardiac α-actin, mlc-2a) lineages (FIG. 5F). Together, these findings indicate that expression of Notch 4 redirects the fate of the BL-CFC from a progenitor with hematopoietic and vascular potential to one with cardiac and vascular potential.

### EXAMPLE 7

Notch Ligand Induces Cardiac Development from Flk-1⁺ Cells.

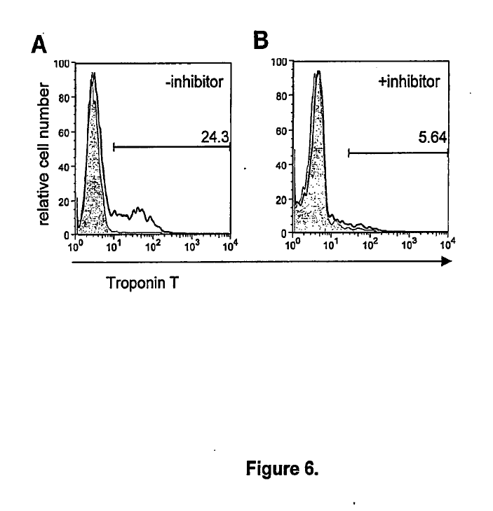

The foregoing examples demonstrate that expression of an activated form of Notch4 can induce cardiac development from hemangioblast mesoderm. To determine if the effect could also be demonstrated by signalling through endogenous Notch receptors, Flk-1⁺ cells from Bry-GFP ES cells were seeded onto OP9 cells that express the Notch ligand Delta-like-1. Following 3 days of culture, areas of contracting cells were detected on the OP9 stromal cells, with approximately 24% of the cells expressing cTnT (FIG. 6A). As with the constitutively activated Notch receptor, cardiornyocyte development on the OP9-DL1 cells was inhibited in the presence of γ-secretase inhibitor, indicating that the effect was specific to Notch signalling (FIG. 6B).

### EXAMPLE 8

Blocking Notch Signalling Inhibits Cardiac Differentiation from the GFP-Bry⁺/Flk-⁻ Population

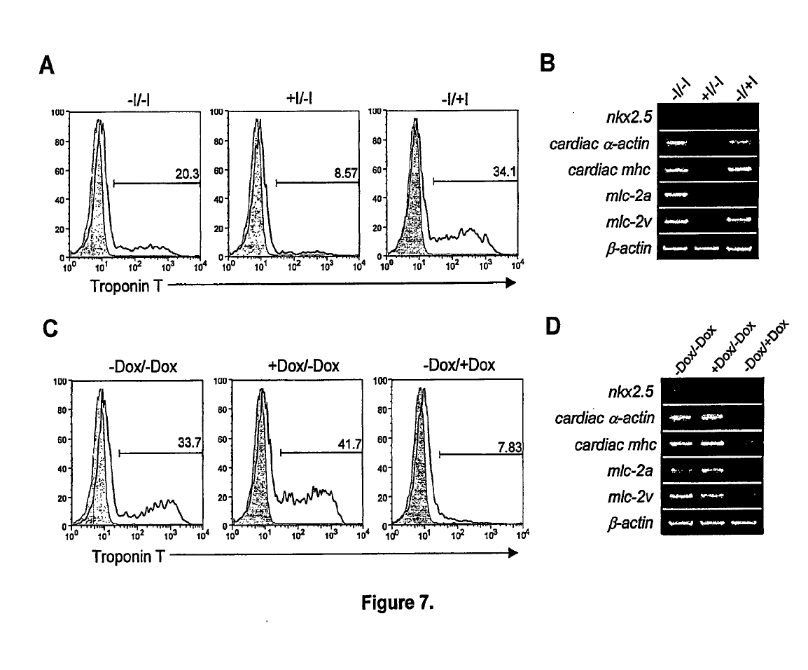

Cardiac potential has been mapped to the Flk-1⁻ fraction of brachytury-expressing mesoderm (GFP-Bry⁺/Flk-1⁻) at early singes of EB development (Kouskoff et al., supra). To investigate the role of Notch4 during cardiac differentiation of this mesoderm, the GFP cDNA was targeted to the brachyury locus of Ainv cells to enable the overexpression of Notch4 in the GFP-Bry⁺/Flk-1⁻ population. The GFP-Bry⁺/Flk-1⁻ fraction was isolated from clay 3.25 EBs derived from the GFP-Bry/Ainv-Notch4 ES cells, reaggregated for 1 day and the resultant aggregates plated in cardiac cultures. γ-Secretase inhibitor or Dox was added to the cells either during the reaggregation step or to the cardiac cultures to further define the stage specific effects of Notch4. Three days following differentiation of the aggregates in the cardiac cultures, the proportion of cTnT-positive cells and the expression of cardiac genes were analyzed (FIG. 7). Blocking Notch signaling by adding γ-secretase inhibitor during the aggregation stage (+I/−I) suppressed the development of cTnT-positive contracting cells and reduced the expression levels of the cardiac genes (FIGS. 7A and 7B), indicating that Notch signaling is critical for cardiomyocyte development from this population. If the inhibitor was added to the cardiac cultures rather than to the aggregates (−I/+I), the proportion of cTnT-expressing population was modestly increased compared to the control (−/−) (FIGS. 7A and 7B). As expected, this population expressed the spectrum of cardiac genes. Induction of Notch4 during the reaggregation stage (+Dox/−Dox) enhanced cardiomyocyte development over that observed in the control cultures (FIG. 7C). In contrast, induction in the cardiac cultures (−Dox/+Dox) inhibited cardiomycyte development as demonstrated by the decrease in cTnT positive cells and the lower expression of the cardiac genes compared with the control culture (−Dox/−Dox) (FIGS. 7C and 7D). This example demonstrates that Notch signaling is essential for the initial stages of cardiomyocyte specification from the ES cell-derived GFP-Bry⁺/Flk-1⁻ population. However, as observed with the Flk-1⁺ population (FIG. 3C), Notch4 expression in the cardiac culture step is inhibitory to the maturation of the cardiac lineage.

### EXAMPLE 9

Blocking Notch Signaling Inhibits Cardiac Differentiation from the Primitive Streak of the Embryo

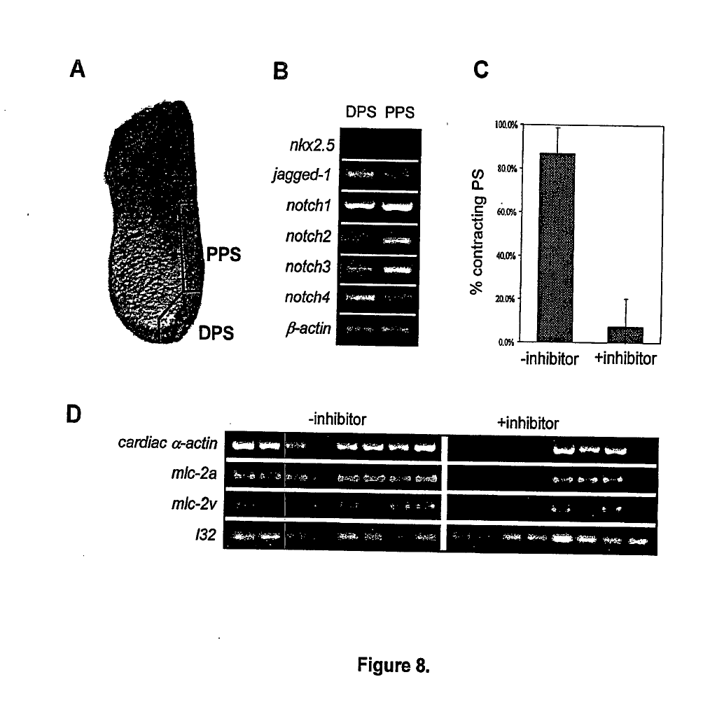

Lineage tracing studies of mouse embryos indicate that the progenitors leading to the cardiac mesoderm of the heart field derive predominantly from the region adjacent to the border of the distal and posterior primitive streaks at embryonic day 7.0-7.5 (E7.0-7.5) (Kinder et al., (1999) Development 126:4691-4701. Analysis of the distal PS (DPS) and posterior PS (PPS) from E7.5 embryos (FIG. 8A) revealed overlapping but distinct expression patterns of the four Notch receptors and the ligand jagged-1 (FIG. 8B). Jagged-1 and Notch1 were expressed in the both regions of the PS. Expression of Notch2 and Notch3 appeared to be higher in the PPS, while Notch4 levels were higher in the DPS. Nkx2.5 was not detected in the PS at this stage of development.

When isolated PPS are plated in the cardiac cultures contracting cardiomyocytes can be detected within 3 to 5 days. To investigate whether Notch signalling is required for the development of the cardiomyocyte lineage from embryo-derived tissues, PPSs were cultured in the presence or absence of γ-secretase inhibitor and then analyzed for the development of contracting cells and for the expression of cardiac genes. In the absence of γ-seoretase inhibitor greater than 80% of the PS explants generated contracting cells. Less than 10% of those cultured with the inhibitor gave rise to these cells (FIG. 8C). Molecular analysis revealed that the contracting cells generated from each PS in the absence of γ-secretase inhibitor expressed cardiac markers, including cardiac α-actin, mlc-2a and mlc-2v (FIG. 8D). In the presence of inhibitor, expression of cardiac genes was inhibited in some but not all of the explants. The lack of reduction of expression in all cultures may be due to the fact that intact pieces of tissue, rather than single cells were assayed making it difficult for the inhibitor to access all cells. The findings from the embryo studies are consistent with those from the ES cell differentiation cultures and indicate that Notch signaling is required for development of the cardiac lineage.

1-28. (canceled)

29. An isolated ES-derived cardiovascular progenitor cells having cardiac and vascular potential.

30. An isolated population of cardiovascular colonies that express cardiac genes, endothelial genes and vascular smooth muscle genes, and do not express gata-1.
